(19) United States
(12) Patent Application Publication
(10) Pub. No.: US 2024/0113409 A1
(43) Pub. Date: Apr. 4, 2024

(54) PROGRAMMABLE ELECTRICAL BYPASS

(71) Applicant: Dario Maximilian Spera, Roma (IT)

(72) Inventor: Dario Maximilian Spera, Roma (IT)

(21) Appl. No.: 18/274,476

(22) PCT Filed: Jan. 27, 2022
(86) PCT No.: PCT/IT2022/050009

§ 371 Date: Jul. 27, 2023

(30) Foreign Application Priority Data
Jan. 27, 2021 (IT) 102021000001541

(51) Int. Cl. H01P 5/16; H05K 5/02

(52) U.S. Cl. CPC H01P 5/16; H05K 5/0217

(57) ABSTRACT

Programmable electrical bypass which finds application in the field of electronics, used for example to orient the electrical charges which generate the electrical field running through the bypass object of the invention. Therefore, the programmable electrical bypass induces, through all the energy expressions of the devices powered by the oriented electric field, electromagnetic and mechanical activities active on biological, organic and inorganic systems. Furthermore, said electromagnetic and mechanical activities allow the correct operation and optimisation of some machinery.

### FIELD OF THE INVENTION

The present invention relates to a programmable electrical bypass which finds application in the field of electronics, used for example to orient the electric charges generating the electric field running through the bypass object of the invention. Therefore, the programmable electrical bypass induces, through all the energy expressions of the devices powered by the oriented electric field, electromagnetic and mechanical activities active on biological, organic and inorganic systems. Furthermore, said electromagnetic and mechanical activities allow the correct operation and optimisation of some machinery.

### STATE OF THE ART

Currently, one of the purposes of the electrical bypasses on the market is to create electrical bridges between different sockets, between sockets and switches and between several switches. Another use involves the application of the electrical bypasses to divert the current flow of a circuit in order to protect a system, machinery or a device, possibly supplied with different voltages or amperes from the main circuit.

In the state of the art, there are no electrical bypasses capable of modifying the action of the electromagnetic field generated by the current flow crossing them, but only connections parallel to the pre-existing electrical circuit capable of diverting the current flow and therefore the electromagnetic field generated.

The need is therefore felt to create a programmable electrical bypass which allows the aforesaid application limits to be overcome.

### SUMMARY OF THE INVENTION

The primary object of the present invention is to create a programmable electrical bypass which allows to orient the electrical charges of which the bypass is composed and those running along it so as to induce, through all the energy expressions of the devices supplied by the oriented electric field, electromagnetic and mechanical activities active on biological, organic and inorganic systems as well as to allow the correct operation and the optimisation of some machinery. Furthermore, the bypass object of the present invention also acts on electronic devices.

Therefore, the present invention aims to achieve the objects discussed above by creating a programmable electrical bypass which, according to claim 1, comprises
at least one conductive electrical connection;
at least one input lead;
at least one output lead;
electrical charges which are equioriented and/or in the same quantum state.

A second aspect of the present invention includes a method for generating active electromagnetic and mechanical activities on biological, organic and inorganic systems by means of a programmable electrical bypass according to the invention which, according to claim 15, comprises the following steps:
providing the programmable electrical bypass with orientation and/or quantum state of the electrical charges suitable for generating the electromagnetic and mechanical activities necessary for their needs;
connecting the programmable electrical bypass to the electrical power supply through the at least one input lead;
connecting the programmable electrical bypass through the at least one output lead to the system on which the necessary electromagnetic and mechanical activities are to be activated or which are to produce the desired activities;
turning on the system;
repeating the previous operations for each new system or for each new programmable electrical bypass to be applied to the system.

The main advantage is the great versatility of the programmable electrical bypass. In fact, said bypass is made according to different embodiments as a function of the type of application.

Furthermore, the programmable electrical bypass with a specific hardware structure can vary its application as a function of the orientation of the electrical charges and/or quantum state, therefore using the same hardware it is possible to vary the application of the bypass object of the invention simply by reprogramming it.

According to an embodiment of the invention, the programmable electrical bypass is an electric cable with input and output leads. Said cable is preferably made of silver or aluminium and optionally, coated with an insulating material.

Advantageously, the bypass of this embodiment can be applied to the power supply circuit of a magnetic resonance to obtain a healing effect of some diseases, to have, for example, a bactericidal and disinfectant effect, to act on the skeletal apparatus by conveying the magnetic fields.

The different effects of the programmable electrical bypass depend on the orientation of the electrical charges and/or their quantum state. These features are defined in the production step of the bypass object of the invention, which is programmed as a function of the application envisaged.

In the form of a cable, the programmable electrical bypass can also be applied in production plants. Depending on the needs, it could be programmed to condition a given metal alloy so as to obtain specific allotropic shapes of a given component. Alternatively, it can be applied to food plants to implement, for example, a germicidal action inside silos or mixers.

In another embodiment of the invention of the programmable electrical bypass, said bypass is applied to a machine for generating plasma, for example for corona treatment.

Alternatively, for example, said bypass is applied to an ozone generating machine.

For example, the bypass object of the invention is applicable to plasma welding machines.

In a further embodiment of the invention, the programmable electrical bypass is a circuit board printed in photolithography. According to this embodiment, the programmable electrical bypass is inserted into portable and non-portable devices such as, by way of example, LCD screens, QLED screens, OLED screens, smartphones and tablets and is programmed to carry out curative actions, for example, anti-inflammatory and analgesic actions in the body of the user of the devices. Alternatively, the programmable electrical bypass is programmed to optimise the operating modes of the device in which it is inserted.

Still according to this embodiment of the invention, the programmable electrical bypass fits into wearable devices such as earphones and 3D viewers and has a curative action, for example, anti-inflammatory and/or analgesic; it can also counteract the harmful effects on the body generated by an electromagnetic field; it can convey and/or exploit the electromagnetic fields generated by the devices.

The bypass object of the invention can be installed in a wearable device such as a heart rate monitor or a smart watch, and suitably programmed to have a vasodilator or vasoconstrictor action.

In light of the different example embodiments of the invention, explanatory but not limiting of the invention, its great adaptability and its infinite programmability according to the needs of the user and of the applications can be seen.

The dependent claims describe preferred embodiments of the invention.

### BRIEF DESCRIPTION OF THE FIGURES

Further features and advantages of the invention will be more apparent in light of the detailed description of two preferred, but not exclusive, embodiments of a programmable electrical bypass, illustrated as a non-limiting example, with the aid of the attached drawing tables in which:

FIG. 1 depicts a perspective view of a first embodiment according to the invention;

FIG. 2 depicts another perspective view of the first embodiment according to the invention;

The same reference numerals in the figures identify the same elements or components.

### DETAILED DESCRIPTION OF THE INVENTION

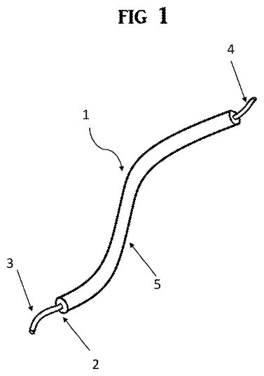

With reference to FIG. 1, a first embodiment of a programmable electrical bypass is depicted, globally indicated with the numerical reference 1. According to this first embodiment, the programmable electrical bypass comprises:
at least one conductive electrical connection, which in this case is at least one electric cable. In fact, in the case of applications of the invention to circuits powered by single-phase current, the programmable electrical bypass comprises a cable, in the case of circuits powered by three-phase current it comprises three cables. The cables are made of conductive material such as silver, aluminium, lithium, magnesium, nickel, copper, alloys thereof or carbonaceous materials.
at least one input lead;
at least one output lead;

The at least one input lead is welded or fixed to the electrical product or to a cable, electrical power supply side; the at least one output lead is welded or fixed to the electrical product or to a cable of a biological, organic, inorganic system, or of machinery or a device.
equioriented electric charges and/or in the same quantum state; said electrical charges are programmed during the production of the programmable electrical bypass with the Holographic Information Transfer (HIT) technique.

Advantageously, the charges can be programmed for multiple purposes both as a function of the programming mode and depending on the conductive material used. This feature makes the programmable electrical bypass a versatile tool which can be adapted to different applications.

For example, the invention is applicable to the circuit of magnetic resonances, production plants, in particular food and pharmaceutical plants.

In the case of pharmaceutical plants, the programmable electrical bypass is installed on the production line and acts through electromagnetic and/or mechanical activities to functionalise drugs, introducing additional curative actions or optimising the existing ones.

This possibility is an advantage: for drug manufacturers, which without changing the production line, through the invention, functionalise existing drugs with new curative properties; for consumers, who benefit from emphasised and optimised curative effects previously given by one or more drugs.

Optionally, the programmable electrical bypass comprises at least one insulating casing which leaves the at least one input lead and the at least one output lead uncovered.

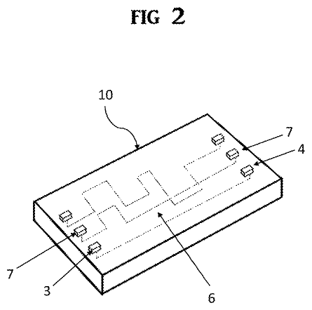

FIG. 2 depicts a second embodiment of a programmable electrical bypass, globally indicated with the numerical reference 10.

According to this second embodiment, the programmable electrical bypass comprises:
at least one conductive electrical connection; which is a graphene printed circuit board in photolithography. This embodiment is suitable for applications in the field of micro and nanoelectronics such as smartphones, tablets, smart watches, etc.
at least one input lead;
at least one output lead;
electrical charges which are equioriented and/or in the same quantum state.

Optionally, this embodiment comprises at least one insulating casing; which is the substrate in which the carbonaceous or metallic material circuit is contained. In this embodiment, said at least one insulating casing is preferably a resin.

Said at least one input lead and at least one output lead are contacts not covered by the at least one insulating casing.

Advantageously, this embodiment allows the application of the bypass of the invention to portable and wearable devices. Furthermore, the invention confers healing properties to said devices, activating, for example, anti-inflammatory and analgesic actions, or optimises the functions thereof by minimising the harmful effect of the electromagnetic fields acting on the body due to the use of said devices.

Another aspect of the present invention includes a method for generating active electromagnetic and mechanical activities on biological, organic and inorganic systems by means of a programmable electrical bypass, comprising the following steps:
providing the programmable electrical bypass with orientation and/or quantum state of the electrical charges suitable for generating the electromagnetic and mechanical activities necessary for their needs;
connecting the programmable electrical bypass to the electrical power supply through the at least one input lead;
connecting the programmable electrical bypass through the at least one output lead to the system on which the necessary electromagnetic and mechanical activities are to be activated or which are to produce the desired activities;
turning on the system;
repeating the previous operations for each new system or for each new programmable electrical bypass to be applied to the system.

1-4. (canceled)

5. A programmable electrical bypass characterized in that it is applicable to circuits powered by single-phase current where at least one conductive connection is unique;
said programmable electrical bypass having a specific hardware structure and being adapted to vary the application thereof according to the orientation of at least one of the electrical charges and the quantum state, therefore using the same hardware to vary the application of the bypass object by reprogramming it, said bypass comprising:
at least one conductive electrical connection;
at least one input lead;
at least one output lead;
electrical charges that are at least one of equioriented and in the same quantum state; and
a circuit printed in photolithography having a weldable input lead and at least one output lead.

6. A programmable electrical bypass according to claim 5, characterized in that the programmable electrical bypass is applicable to three-phase current powered circuits and comprises three conductive connections.

7. A programmable electrical bypass according to claim 5, wherein the circuit is covered with at least one insulated casing and the at least one insulated casing of the circuit is realized in resin.

8. A programmable electrical bypass according to claim 6, wherein the circuit is covered with at least one insulated casing and the at least one insulated casing of the circuit is realized in resin.
